(19) United States
(12) Patent Application Publication
(10) Pub. No.: US 2011/0002907 A1
(43) Pub. Date: Jan. 6, 2011

(54) Methods, Systems, Compositions And Dosage Forms For Diagnosing And Treating Male Infertility

(75) Inventor: Haim Breitbart, Petach Tikva (IL)

Correspondence Address: The Law Office of Michael E. Kondoudis, 888 16th Street, N.W., Suite 800, Washington, DC, 20006, US

(73) Assignee: PEERION MEDICAL TECHNOLOGIES LTD., Zichron Ya'acov (IL)

(21) Appl. No.: 12/919,067

(22) PCT Filed: Feb. 5, 2009
(86) PCT No.: PCT/IL09/00137

§ 371 Date: Aug. 24, 2010

Related U.S. Application Data
(60) Provisional application No. 61/064,278, filed on Feb. 26, 2008.

(51) Int. Cl. A61K 38/46; G01N 33/53; C12N 5/076; A61K 31/661; A61K 33/10; A61K 31/7076; A61K 31/225; A61K 35/48; A61P 15/08

(52) U.S. Cl. 424/94.6; 435/7.21; 435/325; 514/121; 424/717; 514/47; 514/548; 424/559

(57) ABSTRACT

Methods and systems are provided for diagnosing male infertility relating to inadequate production of phosphatidic acid and are complementary to the routine tests, assessing sperm count, motility, viability, head morphology, and white blood cell count. Additional therapeutic methods, compositions and dosage forms are provided for treating male infertility that is related to inadequate production of phosphatidic acid. Such therapeutic approaches involve the use of phosphatidic acid or at least one of its precursors in the sperm intracellular signaling pathway.

### FIELD OF THE INVENTION

The present invention relates to methods, systems, compositions and dosage forms for diagnosing and treating male infertility.

### BACKGROUND OF THE INVENTION

In order to fertilize an egg, mammalian sperm normally need to reside in the female reproductive tract for several hours, during which they undergo a series of biochemical modifications collectively called capacitation. Only capacitated sperm can undergo the acrosome reaction after binding to the egg zona pellucida, a process which enables sperm to penetrate into the egg and fertilize it. Polymerization of globular (G)-actin to filamentous (F)-actin occurs during capacitation, depending on protein kinase A activation, protein tyrosine phosphorylation, and phospholipase D activation. F-actin formation is important for the translocation of phospholipase C from the cytosol to the sperm plasma membrane during capacitation.

Prior to the occurrence of the acrosome reaction, the F-actin undergoes depolymerization, a necessary process that enables the outer acrosomal membrane and the overlying plasma membrane to come into close proximity and fuse. The binding of the capacitated sperm to the zona pellucida induces a fast increase in sperm intracellular calcium and activation of actin, which severs proteins that break down the actin fibers, and allows the acrosome reaction to take place.

Roughly one-third of infertility cases can be attributed to male factors and another one-third to factors that affect women. For the remaining infertile couples, infertility is caused by a combination of problems in both partners (about 13%) or is unexplained (about 10%).

The most common causes of male infertility include azoospermia (no sperm cells are produced) and oligospermia (few sperm cells are produced). Sometimes, sperm cells are malformed or they die before they can reach the egg. In rare cases, male infertility is caused by a genetic disease such as cystic fibrosis or a chromosomal abnormality.

The intra-cellular signaling mechanisms by which phosphatidic acid (PA) is produced and the role of PA within the poorly understood process of sperm capacitation have been examined. In brief, one major role of PA is to mediate the polymerization of G-actin to intracellular filamentous actin (F-actin), which is critical for the process in which capacitated sperm binds to the egg zona pellucida and thereafter, the process of acrosome reaction takes place.

Therefore, an unexplained male infertility can likely be attributed to a failure in the signaling mechanism that is responsible for the production of PA.

In the case that infertility is suspected to be related to the male, a few types of tests are used to assess the quality of male sperm. Five parameters that are frequently used to analyze sperm quality, and which are recommended by the World Health Organization (WHO) are: (1) sperm count; (2) sperm motility; (3) sperm viability; (4) white blood cell (WBC) count; (5) sperm head morphology.

Based on the results from a few, or all, of these abovementioned tests, a selected treatment option is usually selected. Currently available treatment options include: (a) intrauterine insemination (IUI), (b) in vitro fertilization (IVF), and intracytoplasmic sperm injection (ICSI). The following briefly describe these three treatment options:

Intrauterine insemination (IUI): IUI alone is not an effective treatment for male factor infertility. Recent techniques of ovarian stimulation with conjunction with IUI, however, significantly improved success rate of IUI in patients with male factor infertility. Since greater number of oocytes present after ovarian stimulation, the chances of sperm-egg interaction are higher.

IUI is most effective in couples with “cervical factor infertility” indicated with poor postcoital tests. In general, men are considered candidates for IUI if all male factors have been corrected or unexplained infertility is suspected. Severe abnormalities in semen parameters indicate low success rates for IUI, a worse prognosis for IUI being correlated with poor sperm motility. Although IUI has been attempted with sperm count as low as 190,000 motile sperm cells, most IUI pregnancies require more than 10⁶ motile sperm in the specimen. No more than 6 cycles of IUI are recommended since no pregnancies are usually achieved beyond 4-5 cycles of IUI.

Ovarian hyperstimulation is achieved using Clomiphene citrate, Pergonal or Metrodin (by increasing FSH) with subsequent production of a large number of oocytes. A semen specimen, collected by masturbation, is washed from seminal fluid and processed by a Percoll gradient or swim-up technique. Motile sperm are collected and inserted into the uterine cavity via the cervical canal under direct vision using a fine “Tomcat” catheter.

In some cases, preliminary sperm function tests may be considered to assess sperm-egg interaction in vitro. If results of these tests are poor, the couple should be advised of other methods of Assisted Reproduction.

In vitro Fertilization (IVF): Although male factor infertility was considered a contraindication to IVF, fertilization and live births are possible with abnormal sperm. The rates of fertilization with poor quality sperm are significantly lower than sperm from otherwise healthy men, even when sperm are placed directly with oocytes in culture. In addition, the needed concentration of 10⁶ sperm/oocyte cannot be easily obtained from patients with male factor infertility.

The technique of IVF includes down regulation of women's pituitary function with GnRH agonists during the preceding luteal phase followed by controlled ovarian hyperstimulation using FSH. After follicle development is confirmed by transvaginal ultrasound and serial measurement of serum estrogen and progesterone levels, final oocyte maturation is induced with IM injection of hCG. Transvaginal oocyte retrieval is performed under ultrasound guidance.

Seminal fluid processed as described above and preferably containing a concentration of 10⁶ or more sperm per oocyte is used to inseminate the oocyte. In case of successful fertilization embryos are incubated in vitro for 2-3 days to divide up to the 8-cell stage and then transferred back to the uterus. Usually up to 4 embryos may be transferred.

Intracytoplasmic sperm injection (ICSI): This is the most aggressive mechanical micromanipulation technique. It involves direct microinjection of a single sperm into the cytoplasm of the oocyte. ICSI surmounts the last barrier of the oocyte, the oolemma. Since first successful ICSI procedure performed by Palermo et al. in 1993, this technique has gained worldwide acceptance because it almost completely bypasses the problems associated with male factor infertility. The procedure requires only 1 viable sperm per oocyte for injection and is basically independent of sperm quality. Refinement and widespread use of sperm retrieval techniques enables the use of ejaculated as well as epididymal and testicular sperm from patients with severe male factor infertility and azoospermia. Fertilization rates were shown to be independent from female factors, but implantation and pregnancy rates are significantly lower in couples with maternal age above 34 (49%, 23% and 6% respectively at <34, 35-39 and >40 years). In addition, ICSI can cause significant mechanical damage to the oocyte. Rates of oocyte loss have been reported to be 7-14%.

Indications for ICSI include but are not limited to: (1) sperm concentration of <2×10⁶ sperm/cm³, (2) sperm motility <5%, (3) strict criteria normal morphology <4%, (4) only surgically retrieved spermatozoa are available and (5) previous IVF cycles were unsuccessful. Technique of ICSI: Ovarian stimulation for oocyte retrieval is performed according to the protocol for IVF. Thirty-six hours after induction of ovulation with 10000 I.U. hCG, oocytes are retrieved by ultrasound-guided puncture of follicles. Only mature oocytes of metaphase II are used for the injection. Metaphase II oocytes are identified by the presence of extrusion of the first polar body. The microinjection is performed with an injection pipette under a microscope equipped with a micromanipulator, after which the oocyte is stabilized with a holding micropipette. The single sperm is injected head first after the micropipette is pushed through the zona pellucida and oolemma at the 3 o'clock position.

As can be seen from the above discussion, even when male factors are identified as the cause of a couple's inability to conceive, accurate determination which of the numerous possible methods of assisted reproduction is optimal for a particular case depends on an accurate knowledge of the details of the male factors involved. Thus, there is a long-felt need for a simple, rapid, and accurate diagnostic method for enabling the clinician to decide which of the possible procedures should be recommended. In addition, there is a long-felt need for a procedure that can increase the chances of fertilization by increasing the ability of the sperm to undergo capacitation.

### SUMMARY OF THE INVENTION

It is thus one object of this invention to disclose a system for predicting success rates of IUI or IVF comprising (a) means for obtaining a sperm sample; (b) means for determining the level of F-actin in the sperm sample and (c) means for comparing the level of F-actin with at least one predetermined F-actin threshold such that a determination that the level of F-actin is below the predetermined F-actin threshold or above the predetermined F-actin threshold is indicative of low probability of success of the IUI or IVF or high probability of success of the IUI or the IVF, respectively.

Another object of the invention is to disclose means and methods to increase the chances of fertilization by increasing the ability of the sperm to undergo capacitation, in particular by using PA (which is critical for the process of capacitation) or one of its precursors as the active pharmaceutical ingredient.

Another object of the invention is to disclose the system additionally comprising (a) a means for determining the level of hyperactivated motility (HAM) of the sperm sample and (b) means for comparing the level of HAM with at least one predetermined HAM threshold.

It is a core purpose of the invention to provide a determination that the level of F-actin is below the predetermined F-actin threshold and a determination that the level of HAM is below the predetermined HAM threshold or a determination that the level of F-actin is above the predetermined F-actin threshold and a determination that the level of HAM is above the predetermined HAM threshold is indicative of low probability of success of the IUI or the IVF or high probability of success of the IUI or the IVF, respectively.

A further object of the invention is to disclose the means for determining HAM comprising a computer assisted sperm analysis (CASA) system.

A further object of the invention is to disclose the system comprising means for determining the level of total motility (TM) of said sperm sample and means for comparing said level of TM with at least one predetermined TM threshold.

It is a core purpose of the invention to provide a determination that the level of F-actin is below the predetermined F-actin threshold and a determination that the level of TM is below said predetermined TM threshold or a determination that the level of F-actin is above the predetermined F-actin threshold and a determination that the level of TM is above the predetermined TM threshold is indicative of low probability of success of the IUI or the IVF or indicative of high probability of success of the IUI or the IVF, respectively.

A further object of the invention is to disclose the means for determining the level of F-actin in the sperm sample and the means for comparing the level of F-actin with the predetermined threshold comprises an enzyme-linked immuno sorbent assay (ELISA) platform.

A further object of the invention is to disclose a method for predicting success rates of IUI or IVF comprising steps of (a) obtaining a sperm sample; (b) determining the level of F-actin in the sperm sample; (c) comparing the level of F-actin with at least one predetermined F-actin threshold; and (d) determining whether the level of F-actin is below the predetermined F-actin threshold or above the predetermined F-actin threshold; thereby indicating low probability of success of the IUI or the IVF or high probability of success of the IUI or the IVF, respectively.

A further object of the invention is to disclose the method additionally comprising steps of (a) determining the level of hyperactivated motility (HAM) of the sperm sample, (b) comparing the level of HAM with at least one predetermined HAM threshold, and (c) determining that said level of F-actin is below the predetermined F-actin threshold and that the level of HAM is below the predetermined HAM threshold or determining that the level of F-actin is above the predetermined F-actin threshold and determining that the level of HAM is above the predetermined HAM threshold thereby indicating low probability of success of the IUI or the IVF or high probability of success of the IUI or the IVF, respectively.

A further object of the invention is to disclose the method additionally comprising steps for determining HAM by means of a computer assisted sperm analysis (CASA) system.

A further object of the invention is to disclose the method comprising the steps of (a) determining the level of total motility (TM) of the sperm sample; (b) comparing the level of TM with at least one predetermined TM threshold; and (c) determining that the level of F-actin is below the predetermined F-actin threshold and that the level of TM is below the predetermined TM threshold or determining that the level of F-actin is above the predetermined F-actin threshold and determining that the level of TM is above the predetermined TM threshold thereby indicating low probability of success of the IUI or the IVF or high probability of success of the IUI or the IVF, respectively.

A further object of the invention is to disclose the method additionally comprising steps of (a) determining the level of F-actin in the sperm sample and (b) comparing the level of F-acting with the predetermined threshold using an enzyme-linked immuno sorbent assay (ELISA) platform.

A further object of the invention is to disclose a method for treating male infertility comprising the steps of (a) obtaining a sperm sample; and (b) exposing the sperm cells to a composition.

It is a core purpose of the invention to provide the composition comprising at least one signaling event component (SEC) selected from the group consisting of phosphatidic acid (PA), a precursor of PA, an activator of PA, an activator of a precursor of PA, any combination of the above. Exposing the sperm cells to the composition increases the likelihood that the sperm cells will undergo capacitation.

A further object of the invention is to disclose the signaling event component (SEC) that is selected from the group consisting of phospholipase D, sodium bicarbonate, 8-Br-cAMP, and phorbol myristoyl acetate (PMA) or any combination thereof.

A further object of the invention is to disclose the method comprising the additional step of exposing the sperm sample to the therapeutic agent for a period of more than about 3 minutes.

A further object of the invention is to disclose the period of more than about 3 minutes comprising a period of more than about 3 minutes and less than about 20 minutes.

A further object of the invention is to disclose the concentration of phosphatidic acid ranged between about 3 and about 30 μg mL⁻¹.

A further object of the invention is to disclose the concentration of phospholipase D ranged of between about 1 and about 20 IU per milliliter.

A further object of the invention is to disclose the concentration of sodium bicarbonate ranged of between about 10 and about 75 mmol L⁻¹.

A further object of the invention is to disclose the concentration of 8-Br-cAMP ranged of between about 0.2 and about 5 mmol L⁻¹.

A further object of the invention is to disclose the concentration of phorbol myristoyl acetate (PMA) ranged of between about 20 and about 500 ng mL⁻¹.

A further object of the invention is to disclose the additional step of determining the ability of the sperm cells to undergo capacitation under nominal physiological conditions.

A further object of the invention is to disclose the step of determining the ability of the sperm sample to undergo capacitation comprises determining the level of F-actin in the sperm sample.

A further object of the invention is to disclose the step of exposing the sperm cells to a therapeutic agent performed on condition that the step of determining the ability of the sperm cells to undergo capacitation under nominal physiological conditions yields a negative result.

A further object of the invention is to disclose the step of exposing said sperm cells to the composition performed only if level of F-actin is below a predetermined threshold.

A further object of the invention is to disclose a system useful for treating male infertility comprising (a) a means of obtaining sperm and (b) a composition for treating said sperm.

It is a core purpose of the invention to provide the composition comprising at least one signaling event component (SEC) selected from the group consisting of PA, a precursor of PA, an activator of PA, an activator of a precursor of PA, or any combination of the above.

A further object of the invention is to disclose the signaling event component selected from the group consisting of phospholipase D, sodium bicarbonate, 8-Br-cAMP, and phorbol myristoyl acetate (PMA) or any combination thereof.

A further object of the invention is to disclose the system further comprising medium selected from the group consisting of Ham's F10 medium, Whittingham's T6 medium, Quinn's medium, human tubal fluid or any combination thereof.

A further object of the invention is to disclose a composition useful in treating male infertility. The aforesaid composition comprises at least one signaling event component (SEC) selected from the group consisting of PA, a precursor of PA, an activator of PA, an activator of a precursor of PA, or any combination of the above.

A further object of the invention is to disclose the signaling event component (SEC) selected from the group consisting of phospholipase D, sodium bicarbonate, 8-Br-cAMP, and phorbol myristoyl acetate (PMA) or any combination thereof.

A further object of the invention is to disclose the composition further comprising medium selected from the group consisting of Ham's F10 medium, Whittingham's T6 medium, Quinn's medium, human tubal fluid or any combination thereof.

A further object of the invention is to disclose the composition further comprising at least one pharmaceutically acceptable polymer.

A further object of the invention is to disclose the composition adapted for administration in a form selected from the group consisting of solid form, gel form, cream form, capsular form, suppository form, liquid form, spray form and droplet form or any combination thereof.

A further object of the invention is to disclose a method for treating male infertility comprising the steps of (a) obtaining a composition adapted for intravaginal application; the composition comprises at least one signaling event component chosen from the group consisting of (1) phosphatidic acid (PA), (2) a precursor of PA, (3) an activator of PA, (4) an activator of a precursor of PA, (5) any combination of the above; and (b) applying the composition intravaginally; thereby reducing male infertility by assisting the capacitation process of sperm cells.

A further object of the invention is to disclose the composition comprising a signalling event component (SEC) selected from the group consisting of phosphatidic acid (PA), a precursor of PA, an activator of PA, an activator of a precursor of PA, phospholipase D, sodium bicarbonate, 8-Br-cAMP, phorbol myristoyl acetate (PMA).

A further object of the invention is to disclose the composition further comprising medium selected from the group consisting of Ham's F10 medium, Whittingham's T6 medium, Quinn's medium, human tubal fluid or any combination thereof.

A further object of the invention is to disclose the composition further comprising at least one pharmaceutically acceptable polymer.

A further object of the invention is to disclose the form of application of the composition selected from the group consisting of solid form, gel form, cream form, capsular form, suppository form.

A further object of the invention is to disclose a method for selecting a procedure for assisted reproduction to be prescribed comprising the steps of: (a) obtaining a sperm sample; (b) determining the level of F-actin in the sperm sample; (c) comparing the level of F-actin with at least one predetermined F-actin threshold; (d) assessing the quality of the sperm in the sperm sample; and, (e) selecting IUI or IVF or ICSFI if said level of F-actin is above the F-actin threshold and the sperm is of at least medium quality.

It is a core purpose of the invention to provide the selecting of IUI or IVF on condition that the threshold and the sperm are of at least medium quality and the selecting of ICSI is on any other condition.

A further object of the invention is to disclose a method comprising further steps of: (a) determining the level of hyper activated motility (HAM) of the sperm sample; (b) comparing the level of HAM with at least one predetermined HAM threshold; (b) assessing the quality of the sperm in the sperm sample; and, (c) selecting IUI or IVF or ICSI.

It is a core purpose of the invention to provide the selecting of IUI or IVF on condition that the threshold and the sperm are of at least medium quality and the selecting of ICSI is on any other condition.

A further object of the invention is to disclose the step of determining the level of HAM comprising steps of analysing HAM levels with a computer assisted sperm analysis CASA system.

A further object of the invention is to disclose the determining the level of F-actin in said sperm sample further comprising preliminary steps of adding a compound selected from the group consisting of phosphatidic acid (PA), a precursor of PA, an activator of phospholipase D, an activator of a precursor of PA, an inhibitor of PA hydrolysis, an inhibitor of converting PA to phospholipids, or any combination of the above.

### DETAILED DESCRIPTION OF THE INVENTION

It will be apparent to one skilled in the art that there are several embodiments of the invention that differ in detail from the embodiments described herein, without affecting the essential nature thereof, and therefore the invention is not limited by that which is illustrated in the figure and described in the specification, but only as indicated in the accompanying claims, with the proper scope determined only by the broadest interpretation of said claims.

As used hereinafter, the term currently unexplained infertility refers to male infertility that cannot be attributed to azoospermia, oligospermia, physical malformation of sperm cells, genetic disease, or a chromosomal abnormality.

As used hereinafter, the term F-actin refers to intracellular filamentous actin.

As used hereinafter, the term defective action of phospholipase D refers to any mechanism by which the cell fails to produce the products of the action of the enzyme phospholipase D, for example (but not limited to) failure in the signaling leading to its activation, failure of its activation despite proper signaling, failure for properly activated phospholipase D to produce the correct products, or failure of the cell to produce phospholipase D.

The following abbreviations are used hereinafter:

PA refers to phosphatidic acid.

AR refers to the acrosome reaction.

cAMP refers to cyclic adenosine monophosphate.

PKA refers to protein kinase A.

LPA refers to lysophosphatidic acid.

PKC refers to protein kinase C.

PLD refers to phospholipase D.

MAP refers to mitogen activated protein.

ADP refers to adenosine diphosphate.

PIP2 refers to phosphatidylinositol-4,5-bisphosphate.

PLA refers to phospholipase A.

IU refers to International Units (a unit of measure).

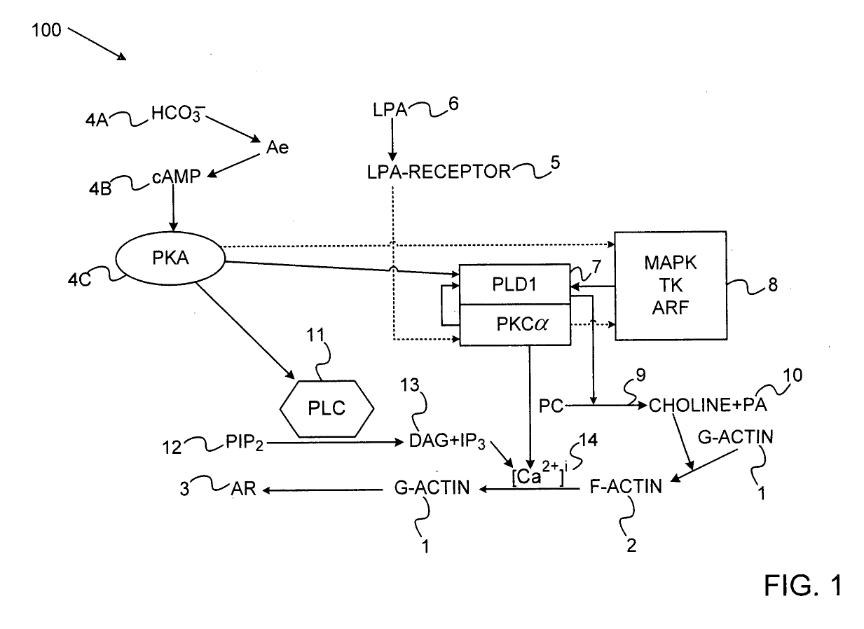

Reference is now made to FIG. 1, which depicts schematically the major signaling events 100 that occur during the remodeling of actin in sperm capacitation and the acrosome reaction (AR). G-actin (1) is polymerized to F-actin (2) during sperm capacitation and the fibers then must undergo depolymerization in order to accomplish the AR (3). Actin polymerization depends on PLD activation, which occurs via the HCO₃²⁻/cAMP/PKA pathway (4a/4b/4c) or via the G-protein coupled receptor (GPCR) (LPA-receptor)/PKC pathway. One of the GPCRs in sperm is LPA-receptor (5) which can be activated by LPA (6), resulting in PKC activation (7) (Garbi et al. 2000) and PLD-dependent actin polymerization (Cohen et al. 2004). MAP-kinase (MAPK), tyrosine kinase (TK), and ADP-ribosylation factor (ARF) (8) are involved in PLD activation, leading to phosphatidyl-choline (PC) hydrolysis to produce phosphatidic acid (PA) (10), which mediates polymerization of G-actin 1 to F-actin 2. The binding of capacitated sperm to the egg zona pellucida activates sperm PLC (11) (Tomes et al. 1996) to hydrolyze PIP2 (12) to diacylglycerol (DAG) and inositol triphosphate (IP3) (13). DAG further activates PKC, and IP3 activates the Ca²⁺ (14) channel in the outer acrosomal membrane resulting in an increase in intracellular Ca²⁺ concentration ([Ca²⁺]ⁱ) (O'Toole et al. 2000). The large increase in [Ca²⁺]ⁱ activates actin-severing proteins to break down F-actin to G-actin and accomplish the AR.

The present invention is based on these biological pathways. It provides a quantitative and/or a semi-quantitative measurement of F-actin. The amount of F-actin is then used as a metric for the level of capacitation of which a single sperm cell, or (in alternative embodiments) a population of sperm cells is capable.

In one of its aspects, the present invention provides a system for diagnosing male infertility arising from defective action of phospholipase D. In a preferred embodiment, the system comprises means for obtaining a sperm sample, means for determining the level of F-actin in the sperm cells within the sperm sample, and means for comparing the level of F-actin with a predetermined threshold. A level of intracellular level of F-actin below the predetermined threshold indicates infertility arising from defective action of phospholipase D. The intracellular level of F-actin may be determined by any method known the art. In an alternative embodiment, the means for determining the level of F-actin and for comparing it to the predetermined threshold comprise an enzyme-linked immuno sorbent assay (“ELISA”) platform of a type well-known to one skilled in the art.

The invention further provides a method for diagnosing male infertility arising from defective action of phospholipase D. This method comprises the steps of collecting a sperm sample, determining the level of F-actin in the sperm cells in the sample, and comparing the level of F-actin within the sperm cells in the sample to a predetermined threshold. If the level is determined to be below the threshold, then the patient is diagnosed as being infertile due to defective action of phospholipase D.

As was discussed above, there are several assisted reproduction methods available and the clinician must choose which of them is optimal for the particular couple coming for treatment. The present invention provides a method for enabling the clinician to determine which of the methods is to be prescribed. This method comprises the steps of obtaining a sperm sample, determining the level of F-actin in the sperm sample, and comparing the F-actin level with a predetermined threshold, as in the diagnostic method. In addition, the quality of the sperm in the sample is assessed. If the F-actin level is above the threshold and the sperm is of at least medium quality (i.e. both conditions are met), then the clinician knows to prescribe IUI or IVF. Since the tests indicate that the sperm have a good ability to undergo capacitation, it makes sense to attempt the less invasive and less costly procedures. If, on the other hand, if either one of those conditions is not met (either the F-actin level is below the threshold or the sperm is of less-than-medium quality or both), then the clinician knows to prescribe ICSI, since in this case, capacitation of the sperm is unlikely or impossible.

The present invention also provides methods and compositions for treating male infertility that is manifested as low potential of sperm cells to undergo capacitation under nominal physiological conditions or that arises from defective action of phospholipidase D. In a preferred embodiment of the invention, the method comprises the steps of obtaining a sperm sample, and then exposing it to a therapeutic agent. The therapeutic agent is one that increases the ability of the sperm cells to undergo capacitation. As PA is critical to the process of capacitation, PA itself can be used as an active pharmaceutical ingredient, to treat sperm of unexplained infertile male subjects. In alternative embodiments, rather than PA itself, a biological precursor of PA, an activator of PA, an activator of a precursor of PA, or a combination of any or all of them, is used as the therapeutic agent. Examples of such therapeutic agents include compounds such as PKA, PKC, PLA and PLD (it is acknowledged and emphasized in this respect that these compounds are given as typical examples only, and not to limit the invention, which includes any compound that can act as a precursor of PA, an activator of PA, or an activator of a precursor of PA).

In alternative embodiments of the method, the therapeutic agent may include inter alia phospholipidase D, sodium bicarbonate, 8-Br-cAMP, phorbol myristoyl acetate (PMA), or a combination of any or all of them.

In a preferred embodiment of the method, the sperm sample is exposed to the therapeutic agent for a period of more than about 3 minutes. In an alternative embodiment of the method, the sperm sample is exposed to the therapeutic agent for a period of between about 3 minutes and about 20 minutes.

In an alternative embodiment of the method, the therapeutic agent is PA and it is administered in a concentration of between about 3 and about 30 μg mL⁻¹. In yet another alternative embodiment of the method, the therapeutic agent is phospholipidase D and it is administered in a concentration of between about 1 and about 20 U mL⁻¹. In yet another alternative embodiment of the method, the therapeutic agent is sodium bicarbonate, and it is administered in a concentration of between about 10 and about 75 mmol L⁻¹. In yet another alternative embodiment of the method, the therapeutic agent is 8-Br-cAMP, and it is administered in a concentration of between about 0.2 and about 5 mmol L⁻¹. In yet another alternative embodiment of the method, the therapeutic agent is PMA, and it is administered in a concentration of between about 20 and about 500 ng mL⁻¹.

Clearly, the effectiveness of the present invention will be in cases of male infertility that manifest themselves as sub-optimal ability for the sperm to undergo capacitation. Thus, alternative embodiments of the method of treatment include as a preliminary step a determination of the ability of the sperm cells to undergo capacitation under nominal physiological conditions. In one such embodiment, this determination is made by measuring the F-actin level in the sperm sample (the measurement can be made according to any one of the procedures well-known to those skilled in the art). In these embodiments, further treatment (i.e. exposing the sperm sample to the therapeutic agent) is only performed if the sperm in the sample are determined to sub-optimally undergo capacitation, e.g. if the level of F-actin is below a predetermined threshold.

It should also be emphasized that the present invention may also be effective for improving the prospects of IVF or IUI procedures in the general population undergoing fertility treatments.

The present invention further discloses various embodiments of a composition for treating human sperm, the treated sperm having an elevated ability to undergo capacitation. This composition includes at least one component chosen from the group consisting of (1) PA, (2) a precursor of PA, (3) an activator of PA, (4) a precursor of an activator of PA, (5) any combination of the above. Various embodiments of the composition include (but are not limited to) PA; phospholipidase D; sodium bicarbonate; 8-Br-cAMP; PMA. In an alternative embodiment of the composition in which it includes PA, the PA is present in the composition in a concentration of between about 3 and about 30 μg mL⁻¹. In an alternative embodiment of the composition in which it includes phospholipidase, the phospholipidase is present in the composition in a concentration of between about 1 and about 20 U mL⁻¹. In an alternative embodiment of the composition in which it includes sodium bicarbonate, the sodium bicarbonate is present in the composition in a concentration of between about 10 and about 75 mmol L⁻¹. In an alternative embodiment of the composition in which it includes 8-Br-cAMP, the 8-Br-cAMP is present in the composition in a concentration of between about 0.2 and about 5 mmol L⁻¹. In an alternative embodiment of the composition in which it includes PMA, the PMA is present in the composition in a concentration of between about 20 and about 500 ng mL⁻¹.

In alternative embodiments of the composition, it includes additional components, including but not limited to Ham's F10 medium; Whittingham's T6 medium; Quinn's medium; and human tubal fluid. Table 1, taken from Tay, J. I.; Rutherford, A. J.; Killick, S. R.; Maguiness, S. D.; Partridge, R. J.; Leese, H. J.; “Human tubal fluid: production, nutrient composition and response to androgenic agents,” Hum. Reprod. 1997, 12, 2451-6, gives the composition of human tubal fluid.

TABLE 1
Mean amino acid concentrations in proliferative (n = 10)
and secretory (n = 7) phase human tubal fluid (mM)

| | Mean | | Mean | | Overall | | |
|---|---|---|---|---|---|---|---|
| Amino acid | proliferative | SEM | secretory | SEM | mean | SEM | P value |
| ASP | 0.017 | 0.004 | 0.041 | 0.018 | 0.027 | 0.008 | 0.097 |
| GLU | 0.071 | 0.019 | 0.111 | 0.033 | 0.091 | 0.019 | 0.275 |
| ASN | 0.063 | 0.053 | 0.028 | 0.025 | 0.046 | 0.018 | 0.439 |
| SER | 0.026 | 0.006 | 0.038 | 0.008 | 0.032 | 0.006 | 0.241 |
| GLN | 0.024 | 0.004 | 0.051 | 0.014 | 0.038 | 0.014 | 0.05 |
| ARG | 0.151 | 0.032 | 0.237 | 0.057 | 0.194 | 0.043 | 0.178 |
| GLY | 0.032 | 0.009 | 0.037 | 0.008 | 0.035 | 0.003 | 0.715 |
| THR | 0.044 | 0.008 | 0.047 | 0.008 | 0.046 | 0.002 | 0.792 |
| ALA | 0.084 | 0.017 | 0.139 | 0.052 | 0.112 | 0.028 | 0.256 |
| TYR | 0.024 | 0.004 | 0.049 | 0.023 | 0.037 | 0.013 | 0.246 |
| TRP | 0.012 | 0.006 | 0.018 | 0.01 | 0.015 | 0.003 | 0.568 |
| MET | 0.012 | 0.004 | 0.014 | 0.003 | 0.013 | 0.001 | 0.662 |
| VAL | 0.022 | 0.006 | 0.032 | 0.005 | 0.026 | 0.004 | 0.269 |
| PHE | 0.016 | 0.005 | 0.018 | 0.003 | 0.017 | 0.001 | 0.714 |
| ILE | 0.017 | 0.005 | 0.02 | 0.002 | 0.019 | 0.002 | 0.568 |
| LEU | 0.05 | 0.014 | 0.067 | 0.013 | 0.059 | 0.009 | 0.398 |
| LYS | 0.042 | 0.009 | 0.061 | 0.016 | 0.052 | 0.01 | 0.261 |

The present invention also discloses a method for the use of PA or PA precursors or activators in the treatment of male infertility in which there is no need for collection of a sperm sample. Instead, the therapeutic agent (i.e. the agent that increases the ability of the sperm to undergo capacitation) is adapted for intravaginal application. The therapeutic agent comprises at least one component chosen from the group comprising (1) PA; (2) a precursor of PA; (3) an activator of PA; (4) a precursor of an activator of PA; (5) any combination of the above, and may be any of the compositions previously listed. The therapeutic agent thus obtained is applied intravaginally (introduced into the female genital tract) prior to sexual intercourse. The therapeutic agent may further include inter alia any pharmaceutically acceptable polymer, and may be applied in a solid form, e.g., incorporated into a solid gelatin capsule; a suppository; a vaginal gel; a vaginal foam; a vaginal cream; or any other method of introducing medication into the female genital tract known in the pharmaceutical art. The specific form into which the therapeutic agent is incorporated will depend on the particular case, and these various forms of applying it are otherwise prepared according to standard methods well-known to those skilled in the art. This therapeutic alternative may be of higher appeal than the standard methods of assisted reproduction to many infertile couples, as it allows them to conceive in a more familiar and friendly surrounding, as opposed to having to undergo the standard intrauterine insemination of either sperm or embryos.

### EXAMPLE

The following clinical trial was made to evaluate sperm capacitation, and is herein disclosed as an exemplary enablement of the invention.

The accompanying table describes the result of a clinical trial performed at a fertility clinic in which 36 male subjects participated. A semen sample was taken from each of the subjects and divided into two aliquots.

The subjects were divided into three groups: (1) Low Acrosome Reaction Group (“Low AR”), in which between 0.9 and 9% of the sperm cells underwent AR, (2) Medium Acrosome Reaction (“Medium AR”) Group, in which between 10 and 19% of the sperm underwent AR, and (3) High Acrosome Reaction (“High AR”) Group, in which between 20 and 40% of the sperm cells underwent AR.

One objective of the above-mentioned clinical trial was to explore the possible correlation between the process of AR and an increase of more than 10% in the amount of F-actin. The percentage of cells that underwent AR was measured as a function of the percentage of cells that underwent a rise of more than 10% in the amount of F-actin in the overall sperm population per subject under simulated nominal physiological conditions.

Results of this comparison are presented in Table 2, showing that within the Low AR groups, only 43% of the sperm cells demonstrated an increase of more than 10% in F-actin, while 63% of sperm cells belonging to the Medium AR Group demonstrated a >10% increase in F-actin and 75% of sperm cells belonging to the High AR Group demonstrated >10% increase in F-actin under simulated nominal physiological conditions.

Another objective of the above-mentioned clinical trial was to determine the effect of exogenous PA on human sperm. Results of this experiment are presented in Table 2, showing that within the Low AR group, addition of exogenous PA caused a rise of 29% in the proportion of sperm cells demonstrating an increase of >10% in F-actin, while in Medium and High AR groups, exogenous PA caused a 50% increase in the proportion of the sperm cells demonstrating an increase of >10% in F-actin. These results suggest that a short incubation with PA causes higher increase in F-actin levels in cells with higher AR rates.

TABL

[symbol]

| | % of induced acrosome | | | |
|---|---|---|---|---|
| | reacted sperm cells | | | |
| | 0.9-9% | 10-19% | 20-40% | |
| | (7 cases) | (22 cases) | (7 cases) | Comments |
| >10% increase | 43% | 63% | 75% | In most cases good |
| in F-actin | | | | correlation is found between |
| | | | | the increase of the AR and F- |
| | | | | actin during sperm |
| | | | | capacitation. |
| PA added for 5 min. | 29% increase | 50% increase | 50% increase | Short incubation with PA |
| | | | | causes higher increase in F- |
| | | | | actin levels in cells with |
| | | | | higher AR rate. |

[symbol]

indicates data missing or illegible when filed

Introduction:

The diagnosis of male infertility mostly relies on microscopic assays of semen quality including sperm concentration, motility and morphology. None of these parameters addresses sperm function and their clinical value in predicting fertility. Capacitation is an essential process for sperm-egg binding, acrosome reaction and egg penetration and its biochemistry and signaling processes are in the front of the research in the fertilization field. Previous assays for sperm capacitation, acrosome reaction, hyperactivated motility and protein tyrosine phosphorylation are difficult to be routinely performed in IVF, expensive and occasionally inconclusive. Sperm capacitation finally leads to actin-polymerization which can be measured based on the ability of the sperm to polymerize G-actin to F-actin.

The object of this clinical trial was to evaluate sperm capacitation by measuring actin-polymerization in semen concurrently used for insemination in a human IVF system.

Materials and Methods: From May 2008 and November 2008 IVF or IVF/ICSI was done in 25 patients as part of the regular IVF service at Assaf Harofeh IVF unit. A 0.5 ml of the sperm sample, used for insemination in IVF, was also checked for capacitation assays: the assay for actin-polymerization was based on the separation between G-actin and F-actin and their quantitative determination, using western blotting with specific antibodies, confirmed by cytochemistry, using phalloidin as a specific dye for F-actin determination. Hyperactivated motility was determined by the CASA computerized system. Acrosome reaction was measured using the lectin FITC-PNA. Only male patients with normal sperm parameters were allocated to this study. IVF alone was performed when a previous pregnancy was achieved and/or when there was previous evidence for fertilization in IVF. IVF/ICSI was done in first IVF cycles. Fertilization rate was correlated with the results of sperm capacitation assays.

Results: The mean age of the male patients was 33±4.5y and of the female . . . y. The mean volume of sperm was 3±0.6 ml (range 2-5), sperm concentration 66±17 million/ml (range 20-50), sperm motility 56±21.2%. (range 30-50) and sperm morphology 7.4±7.7% (range 3-16, Kruger). In 67% of the cycles IVF/ICSI was performed and in the remaining third IVF alone was done. A mean number of 15±10.6 oocytes (range 4-27) were retrieved of them a mean number of 8.7±10.6 (range 3-17oocytes) were exposed to IVF. Our preliminary data with 25 patients revealed a relatively good correlation (correlation coefficient=˜0.6) between the rate of IVF success and the actin polymerization rate at the the actin polymerization rate at the the actin polymerization rate at the end of in vitro capacitation. Linear regression (R) analysis for the comparison between actin polymerization and IVF success rate revealed R=0.512 which was enhanced to 0.728 by adding the hyperactivated motility data to the calculation. The R-square value (0.53) indicate a predictive value of 53%. No correlation was found between IVF rates and the spontaneous or ionophore induced acrosome reaction rates.

Conclusions: Among the IVF patients, a notably high % of sharing cycles (IVF/ICSI were done, representing the apprehension of lack of fertilizations in “IVF only” cycles. This emphasizes the need of the clinician for a reliable functional test that can predict fertilization rates in IVF. A notably good correlation (correlation coefficient=˜0.6) between the rate of IVF success and the actin polymerization rate at the end of in vitro capacitation. The assay is very easy to be performed in each fertility clinic and we are now in the process of developing a simple Elisa kit for this determination.

Statistical Analysis:

1) The values for the pearson correlation are: Actin polymerization at 3 h vs. IVF: 0.535 (p=0.01). HAM at 5 min vs. IVF: 0.432 (p=0.057).

2) Actin polymerization rate together with HAM at 5 min gives a good predictive value for IVF successes (IVF rate >50% defined as success and <50% as failure). Low values of the two (actin polymerization at 3 h <40% and HAM 5 min <10%) indicate 100% failure in IVF (meaning 100% chance to see <50% success in IVF). Low actin polymerization <40% increase from zero time but HAM value >10% or vice versa provide a 83% prediction of IVF (meaning 83% chance to see >50% success in IVF). Actin polymerization >40% and HAM >10% gives 100% prediction of IVF success (meaning 100% chance to see >50% success in IVF).

3) Taking together actin polymerization at 3 h+HAM at 5 min+total motility in 3 h gives 100% prediction of IVF success (meaning 100% chance to see >50% success in IVF rate).

4) By linear regression analysis the R value for the positive prediction by actin polymerization and IVF is 0.512 while when actin polymerization+HAM were taken together they gave R value of 0.728 for positive prediction of IVF rate, and the R square (0.53) gives 53% prediction.

It is herein acknowledged that several of the above embodiments of the invention utilize means and methods of determining total motility (TM) and hyperactive motility (HAM), but that any measure of motility may be determined and may be utilized in the realization of the invention.

Furthermore, it is herein acknowledged that several embodiments of the invention include adding a compound selected from the group consisting of phosphatidic acid (PA), a precursor of PA, an activator of phospholipase D, an activator of a precursor of PA, an inhibitor of PA hydrolysis, an inhibitor of converting PA to phospholipids, or any combination of the above useful for expediting capacitation.

Moreover, it is herein acknowledged that, in some embodiments of the invention, determining the level of F-actin in said sperm sample further comprises preliminary steps of adding a compound selected from the group consisting of phosphatidic acid (PA), a precursor of PA, an activator of phospholipase D, an activator of a precursor of PA, an inhibitor of PA hydrolysis, an inhibitor of converting PA to phospholipids, or any combination of the above.

1-50. (canceled)

51. A system for predicting success rates of IUI or IVF comprising:
a. means for obtaining a sperm sample;
b. means for determining the level of F-actin in said sperm sample, and;
c. means for comparing said level of F-actin with at least one predetermined F-actin threshold;
such that a determination that said level of F-actin is below said predetermined F-actin threshold or above said predetermined F-actin threshold is indicative of low probability of success of said IUI or IVF or high probability of success of said IUI or IVF, respectively.

52. The system according to claim 51, wherein said system additionally comprises:
a. means for determining the level of hyperactivated motility (HAM) of said sperm sample, and;
b. means for comparing said level of HAM with at least one predetermined HAM threshold;
wherein a determination that said level of F-actin is below said predetermined F-actin threshold and a determination that said level of HAM is below said predetermined HAM threshold or a determination that said level of F-actin is above said predetermined F-actin threshold and a determination that said level of HAM is above said predetermined HAM threshold is indicative of low probability of success of said IUI or IVF or high probability of success of said IUI or IVF, respectively; further wherein said means for determining HAM comprises a computer assisted sperm analysis (CASA) system.

53. The system according to claim 51 wherein said system comprises:
a. means for determining the level of total motility (TM) of said sperm sample, and;
b. means for comparing said level of TM with at least one predetermined TM threshold;
wherein a determination that said level of F-actin is below said predetermined F-actin threshold and a determination that said level of TM is below said predetermined TM threshold or a determination that said level of F-actin is above said predetermined F-actin threshold and a determination that said level of TM is above said predetermined TM threshold is indicative of low probability of success of said IUI or IVF or indicative of high probability of success of said IUI or IVF, respectively.

54. The system according to claim 51, wherein said system additionally comprises:
a. means for determining the level of motility of said sperm sample, and;
b. means for comparing said level of motility with at least one predetermined motility threshold;
wherein a determination that said level of F-actin is below said predetermined F-actin threshold and a determination that said level of motility is below said predetermined motility threshold or a determination that said level of F-actin is above said predetermined F-actin threshold and a determination that said level of motility is above said predetermined motility threshold is indicative of low probability of success of said IUI or IVF or high probability of success of said IUI or IVF, respectively.

55. The system according to claim 51, wherein said means for determining the level of F-actin in said sperm sample and said means for comparing said level of F-actin with said predetermined threshold comprises an enzyme-linked immuno sorbent assay (ELISA) platform.

56. The system as in claim 51, wherein said system additionally comprises a compound selected from the group consisting of phosphatidic acid (PA), a precursor of PA, an activator of phospholipase D, an activator of a precursor of PA, an inhibitor of PA hydrolysis, an inhibitor of converting PA to phospholipids, or any combination of the above useful for expediting capacitation, and wherein the addition of said compound is adapted to allow prompting said determining the level of F-actin in sperm sample.

57. A method for predicting success rates of IUI or IVF comprising steps of:
a. obtaining a sperm sample;
b. determining the level of F-actin in said sperm sample, and;
c. comparing said level of F-actin with at least one predetermined F-actin threshold;
d. determining whether said level of F-actin is below said predetermined F-actin threshold or above said predetermined F-actin threshold;
thereby indicating low probability of success of said IUI or IVF or high probability of success of said IUI or IVF, respectively.

58. The method according to claim 57, wherein said method additionally comprises steps of:
a. determining the level of hyperactivated motility (HAM) of said sperm sample;
b. comparing said level of HAM with at least one predetermined HAM threshold, and;
c. determining that said level of F-actin is below said predetermined F-actin threshold and that said level of HAM is below said predetermined HAM threshold or determining that said level of F-actin is above said predetermined F-actin threshold and determining that said level of HAM is above said predetermined HAM threshold thereby indicating low probability of success of said IUI or IVF or high probability of success of said IUI or IVF, respectively.

59. The method according to claim 58, wherein said method additionally comprises at least one step selected from (a) determining HAM by means of a computer assisted sperm analysis (CASA) system; and (b) determining the level of F-actin in said sperm sample and comparing said level of F-acting with said predetermined threshold using an enzyme-linked immuno sorbent assay (ELISA) platform; or any combination thereof.

60. The method according to claim 57, wherein said method comprises steps of:
a. determining the level of total motility (TM) of said sperm sample, and;
b. comparing said level of TM with at least one predetermined TM threshold;
c. determining that said level of F-actin is below said predetermined F-actin threshold and that said level of TM is below said predetermined TM threshold or determining that said level of F-actin is above said predetermined F-actin threshold and determining that said level of TM is above said predetermined TM threshold thereby indicating low probability of success of said IUI or IVF or high probability of success of said IUI or IVF, respectively.

61. The method according to claim 57, wherein said method comprises steps of:
a. determining the level of motility of said sperm sample, and;
b. comparing said level of motility with at least one predetermined motility threshold;
c. determining that said level of F-actin is below said predetermined F-actin threshold and that said level of motility is below said predetermined motility threshold or determining that said level of F-actin is above said predetermined F-actin threshold and determining that said level of motility is above said predetermined motility threshold thereby indicating low probability of success of said IUI or IVF or high probability of success of said IUI or IVF, respectively.

62. The method according to claim 57, wherein said system additionally comprises a step of adding a compound selected from the group consisting of phosphatidic acid (PA), a precursor of PA, an activator of phospholipase D, an activator of a precursor of PA, an inhibitor of PA hydrolysis, an inhibitor of converting PA to phospholipids, or any combination of the above useful for expediting capacitation, wherein said step of adding a compound is adapted to allow prompting said determining the level of F-actin in sperm sample.

63. A method for treating male infertility, comprising the steps of:
a. obtaining a sperm sample, and;
b. exposing said sperm cells to a composition, wherein said composition comprises at least one signaling event component (SEC) selected from the group consisting of phosphatidic acid (PA), a precursor of PA, an activator of PA, an activator of a precursor of PA, any combination of the above;
wherein exposing said sperm cells to said composition increases the likelihood that said sperm cells will undergo capacitation.

64. The method as in claim 63, additionally comprising at least one step selected from (a) selecting said signaling event component (SEC) from the group consisting of phospholipase D, sodium bicarbonate, 8-Br-cAMP, and phorbol myristoyl acetate (PMA) or any combination thereof; or (b) exposing said sperm sample to said SEC for a period of more than about 3 minutes; (c) exposing said sperm sample to said SEC for a period of more than about 3 minutes and less than about 20 minutes.

65. The method as in claim 63, wherein the concentration of phosphatidic acid is in the range between about 3 and about 30 μg mL⁻¹; further wherein the concentration of phospholipase D is in the range of between about 1 and about 20 IU per milliliter; further wherein the concentration of sodium bicarbonate is in the range of between about 10 and about 75 mmol L⁻¹; further wherein the concentration of 8-Br-cAMP is in the range of between about 0.2 and about 5 mmol L⁻¹; further wherein the concentration of phorbol myristoyl acetate (PMA) is in the range of between about 20 and about 500 ng mL⁻¹.

66. The method as in claim 63, further comprising step of determining the ability of said sperm cells to undergo capacitation under nominal physiological conditions; further wherein said step of determining the ability of said sperm cells to undergo capacitation comprises determining the level of F-actin in said sperm sample.

67. The method as in claim 66, additionally comprising at least one step selected from (a) exposing said sperm cells to a therapeutic agent on condition that said step of determining the ability of said sperm cells to undergo capacitation under nominal physiological conditions yields a negative result; or (b) exposing said sperm cells to said composition is performed only if level of F-actin is below a predetermined threshold.

68. A system useful for treating male infertility comprising:
a. means of obtaining sperm, and;
b. composition for treating said sperm;
wherein said composition comprises at least one signaling event component (SEC) selected from the group consisting of PA, a precursor of PA, an activator of PA, an activator of a precursor of PA, phospholipase D, sodium bicarbonate, 8-Br-cAMP, and phorbol myristoyl acetate (PMA) or any combination of the above.

69. The system according to claim 68, wherein the concentration of phosphatidic acid is in the range of between about 3 and about 30 μg mL⁻¹; further wherein the concentration of phospholipase D is in the range of between about 1 and about 20 IU mL⁻¹; further wherein the concentration of sodium bicarbonate is in the range of between about 10 and about 75 mmol L⁻¹; further wherein the concentration of 8-Br-cAMP is in the range of between about 0.2 and 5 mmol L⁻¹; further wherein the concentration of phorbol myristoyl acetate (PMA) is in the range of between about 20 and about 500 ng mL⁻¹.

70. The system according to claim 68, further comprising medium selected from the group consisting of Ham's F10 medium, Whittingham's T6 medium, Quinn's medium, human tubal fluid or any combination thereof.

71. A composition useful in treating male infertility wherein said composition comprises at least one signaling event component (SEC) selected from the group consisting of PA, a precursor of PA, an activator of PA, an activator of a precursor of PA, or any combination of the above.

72. The composition according to claim 71, wherein said signaling event component (SEC) is selected from the group consisting of phospholipase D, sodium bicarbonate, 8-Br-cAMP, and phorbol myristoyl acetate (PMA) or any combination thereof.

73. The composition according to claim 71, wherein said composition further comprises at least one selected from (a) medium selected from the group consisting of Ham's F10 medium, Whittingham's T6 medium, Quinn's medium, human tubal fluid or any combination thereof; or (b) at least one pharmaceutically acceptable polymer; or any combination thereof.

74. The composition according to claim 71, wherein said composition is adapted for administration in a form selected from the group consisting of solid form, gel form, cream form, capsular form, suppository form, liquid form, spray form and droplet form or any combination thereof.

75. A method for treating male infertility, comprising the steps of:
a. obtaining a composition adapted for intravaginal application wherein said composition comprises at least one signaling event component chosen from the group consisting of (1) phosphatidic acid (PA), (2) a precursor of PA, (3) an activator of PA, (4) an activator of a precursor of PA, (5) any combination of the above;
b. applying said composition intravaginally;
thereby reducing male infertility by assisting the capacitation process of sperm cells.

76. The method of claim 75, wherein said composition comprises a signalling event component (SEC) selected from the group consisting of phosphatidic acid (PA), a precursor of PA, an activator of PA, an activator of a precursor of PA, phospholipase D, sodium bicarbonate, 8-Br-cAMP, phorbol myristoyl acetate (PMA).

77. The method of claim 75, wherein said composition further comprises at least one selected from (a) medium selected from the group consisting of Ham's F10 medium, Whittingham's T6 medium, Quinn's medium, human tubal fluid or any combination thereof; or (b) at least one pharmaceutically acceptable polymer; or any combination thereof.

78. The method of claim 75, wherein the form of application of said composition is selected from the group consisting of solid form, gel form, cream form, capsular form, suppository form.

79. A method for selecting a procedure for assisted reproduction to be prescribed, comprising the steps of:
a. obtaining a sperm sample;
b. determining the level of F-actin in said sperm sample;
c. comparing said level of F-actin with at least one predetermined F-actin threshold;
d. assessing the quality of the sperm in said sperm sample, and;
e. selecting IUI or IVF or ICSFI if said level of F-actin is above said F-actin threshold and said sperm is of at least medium quality;
wherein said selecting of IUI or IVF is on condition that said threshold and said sperm is of at least medium quality and said selecting of ICSI is on any other condition.

80. The method according to claim 79, further comprising steps of:
a. determining the level of hyper activated motility (HAM) of said sperm sample;
b. comparing said level of HAM with at least one predetermined HAM threshold;
c. assessing the quality of the sperm in said sperm sample, and;
d. selecting IUI or IVF or ICSI
wherein said selecting of IUI or IVF is on condition that said threshold and said sperm is of at least medium quality and said selecting of ICSI is on any other condition.

81. The method according to claim 80 wherein said step of determining the level of HAM comprises steps of analysing HAM levels with a computer assisted sperm analysis (CASA) system; further wherein said step of determining the level of F-actin in said sperm sample further comprises preliminary steps of adding a compound selected from the group consisting of phosphatidic acid (PA), a precursor of PA, an activator of phospholipase D, an activator of a precursor of PA, an inhibitor of PA hydrolysis, an inhibitor of converting PA to phospholipids, or any combination of the above.

82. The method according to claim 79 comprising further steps of:
a. determining the level of motility of said sperm sample;
b. comparing said level of motility with at least one predetermined motility threshold;
c. assessing the quality of the sperm in said sperm sample, and;
d. selecting IUI or IVF or ICSI;
wherein said selecting of IUI or IVF is on condition that said threshold and said sperm is of at least medium quality and said selecting of ICSI is on any other condition.
